(19) United States
(12) Patent Application Publication
(10) Pub. No.: US 2004/0048886 A1
(43) Pub. Date: Mar. 11, 2004

(54) Pharmaceutical compositions based on new anticholinergics and NK1 receptor antagonists

(75) Inventors: Christopher J. M. Meade, Bingen (DE); Michel Pairet, Biberach (DE); Michael P. Pieper, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/614,362

(22) Filed: Jul. 7, 2003

Related U.S. Application Data
(60) Provisional application No. 60/407,758, filed on Sep. 3, 2002.

(30) Foreign Application Priority Data
Jul. 9, 2002 (DE) 102 30 750.4

(51) Int. Cl. A61K 31/4745

(52) U.S. Cl. 514/291

(57) ABSTRACT

The present invention relates to novel pharmaceutical compositions based on new anticholinergics and NK₁ receptor antagonists, processes for preparing them and their use in the treatment of respiratory diseases.

### RELATED APPLICATIONS

Benefit of U.S. Provisional Application Serial No. 60/407,758, filed on Sep. 3, 2002 is hereby claimed.

### FIELD OF THE INVENTION

The present invention relates to novel pharmaceutical compositions based on new anticholinergics and NK₁ receptor antagonists, processes for preparing them and their use in the treatment of respiratory complaints.

### DESCRIPTION OF THE INVENTION

The present invention relates to novel pharmaceutical compositions based on new anticholinergics and NK₁ receptor antagonists, processes for preparing them and their use in the treatment of respiratory complaints.

Surprisingly, an unexpectedly beneficial therapeutic effect, particularly a synergistic effect can be observed in the treatment of inflammatory and/or obstructive diseases of the respiratory tract if one or more, preferably one, new anticholinergic of formula 1 is used with one or more, preferably one, NK₁ receptor antagonist 2. In view of this synergistic effect the pharmaceutical combinations according to the invention can be used in smaller doses than would be the case with the individual compounds used in monotherapy in the usual way.

The combinations of active substances according to the invention are surprisingly characterised both by a rapid onset of activity and also by a long-lasting duration of activity. This is very important to the patient's feeling of well-being, as on the one hand they experience a rapid improvement in their condition once the combination has been administered and on the other hand the drug need only be taken once a day, thanks to its long-lasting effects.

These effects are observed both when the active substances are administered simultaneously within a single active substance formulation and also when the two active substances are administered successively in separate formulations. It is preferable according to the invention to administer the two active ingredients simultaneously in a single formulation.

Within the scope of the present invention the anticholinergics used are the salts of formula 1

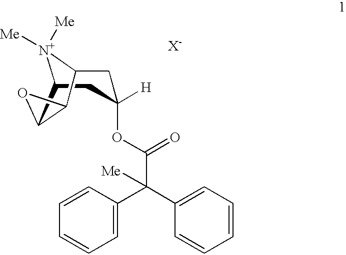

wherein

X—denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate.

Preferably, the salts of formula 1 are used wherein

X—denotes an anion with a single negative charge selected from the group consisting of chloride, bromide, 4-toluenesulphonate and methanesulphonate, preferably bromide.

Most preferably, the salts of formula 1 are used wherein

X—denotes an anion with a single negative charge selected from the group consisting of chloride, bromide and methanesulphonate, preferably bromide.

Particularly preferred according to the invention is the salt of formula 1 wherein

X—denotes bromide.

The salts of formula 1 are known from International Patent Application WO 02/32899.

Within the scope of the present patent application, an explicit reference to the pharmacologically active cation of formula

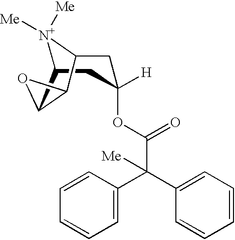

can be recognised by the use of the designation 1′. Any reference to compounds 1 naturally includes a reference to the cation 1′.

Any reference within the scope of the present invention to the salts 1 which may be used according to the invention also includes any hydrates and solvates of these compounds which may be obtainable.

Within the scope of the present invention the term NK₁ receptor antagonists (hereinafter 2) preferably denotes those compounds which are selected from among N-[2-(3,5-bis-trifluoromethyl-phenyl)-ethyl]-2-{4-cyclopropylmethyl-piperazin-1-yl}-N-methyl-2-phenyl-acetamide (BIIF 1149), CP-122721, FK-888, NKP 608C, NKP 608A, CGP 60829, SR 48968 (Saredutant), SR 140333 (Nolpitantium besilate/chloride), LY 303 870 (Lanepitant), MEN-11420 (Nepadutant), SB 223412, MDL-105172A, MDL-103896, MEN-11149, MEN-11467, DNK 333A, SR-144190, YM-49244, YM-44778, ZM-274773, MEN-10930, S-19752, Neuronorm, YM-35375, DA-5018, Aprepitant (MK-869), L754030, CJ-11974, L-758298, DNK-33A, 6b-I, CJ-11974, TAK-637, GR 205171 and the arylglycinamide derivatives of general formula 3

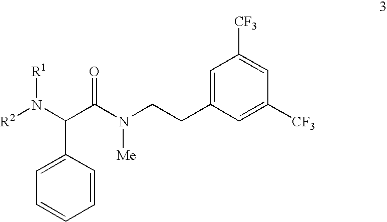

wherein

R¹ and R² together with the N to which they are bound form a ring of formula

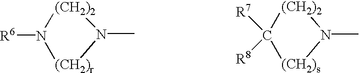

wherein r and s are 2 or 3;

R⁶ denotes H, —C₁-C₅-alkyl, C₃-C₅-alkenyl, propynyl, hydroxy(C₂-C₄)alkyl, methoxy(C₂-C₄)alkyl, di(C₁-C₃)alkylamino(C₂-C₄)alkyl, amino(C₂-C₄)alkyl, amino, di(C₁-C₃)alkylamino, monofluoro- to perfluoro(C₁-C₂)alkyl, N-methylpiperidinyl, pyridyl, pyrimidinyl, pyrazinyl or pyridazinyl,

R⁷ has one of the meanings (a) to (d),

(a) hydroxy

(b) 4-piperidinopiperidyl,

(c)

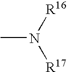

wherein R¹⁶ and R¹⁷ independently of each other denote H, (C₁-C₄)alkyl, (C₃-C₆)cycloalkyl, hydroxy(C₂-C₄)alkyl, dihydroxy(C₂-C₄)alkyl, (C₁-C₃)alkoxy(C₂-C₄)alkyl, phenyl(C₁-C₄)alkyl or di(C₁-C₃)alkylamino(C₂-C₄)alkyl,

R⁸ denotes H,

optionally in the form of the enantiomers and mixtures of enantiomers thereof, optionally in the form of the racemates thereof.

The abovementioned compounds of formula 3 are known for example from International Patent Applications WO 96/32386, WO 97/32865 and WO 02/32865, to which reference is hereby made in their entirety.

Preferably, the compound 2 is selected from among BIIF 1149, CP-122721, CGP 60829, MK-869, CJ-11974, GR 205171 and the arylglycinamide derivatives of general formula 3, wherein

R¹ and R² together with the N to which they are bound form a ring of formula

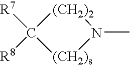

wherein s is 2 or 3;

R⁷ denotes a group

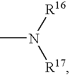

wherein R¹⁶ and R¹⁷ independently of each other denote H, (C₁-C₄)alkyl, (C₃-C₆)cycloalkyl, hydroxy(C₂-C₄)alkyl, dihydroxy(C₂-C₄)alkyl, (C₁-C₃)alkoxy(C₂-C₄)alkyl, phenyl(C₁-C₄)alkyl or di(C₁-C₃)alkylamino(C₂-C₄)alkyl,

R⁸ denotes H,

optionally in the form of the enantiomers and mixtures of enantiomers thereof and optionally in the form of the racemates thereof.

Particularly preferably, the compound 2 is selected from among BIIF1149 and the arylglycinamide derivatives of general formula 3, wherein

R¹ and R² together with the N to which they are bound form a ring of formula

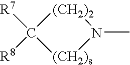

wherein s is 2 and

R⁷ denotes a group

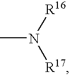

wherein R¹⁶ and R¹⁷ independently of each other denote H, (C₁-C₄)alkyl, (C₃-C₆)cycloalkyl, hydroxy(C₂-C₄)alkyl or dihydroxy(C₂-C₄)alkyl,

R⁸ denotes H,

optionally in the form of the enantiomers and mixtures of enantiomers thereof and optionally in the form of the racemates thereof.

Most particularly preferred as compounds of formula 2 are N-[2-(3,5-bis-trifluoromethyl-phenyl)-ethyl]-2-{4-[(3-hydroxy-propyl)-methyl-amino]piperidin-1-yl}-N-methyl-2-phenyl-acetamide, N-[2-(3,5-bis-trifluoromethyl-phenyl)-ethyl]-2-[4-(2-hydroxy-1-hydroxymethyl-ethylamino)-piperidin-1-yl]-N-methyl-2-phenylacetamide, N-[2-(3,5-bis-trifluoromethyl-phenyl)-ethyl]-2-[4-(cyclopropylmethyl-methyl-amino)-piperidin-1-yl]-N-methyl-2-phenyl-acetamide, N-[2-(3,5-bis-trifluoromethyl-phenyl)-ethyl]-2-{4-[(2-hydroxy-ethyl)-(3-hydroxypropyl)-amino]-piperidin-1-yl}-N-methyl-2-phenyl-acetamide and N-[2-(3,5-bis-trifluoromethyl-phenyl)-ethyl]-2-{4-[cyclopropylmethyl-(3-hydroxy-propyl)-amino]-piperidin-1-yl}-N-methyl-2-phenyl-acetamide, optionally in the form of the enantiomers and mixtures of enantiomers thereof and optionally in the form of the racemates thereof.

Of particular importance is N-[2-(3,5-bis-trifluoromethyl-phenyl)-ethyl]-2-[4-(2-hydroxyl-1-hydroxymethyl-ethylamino)-piperidin-1-yl]-N-methyl-2-phenylacetamide, optionally in the form of its enantiomers, preferably in the form of its (S)-enantiomer, optionally in the form of the mixtures of enantiomers thereof, and optionally in the form of the racemates thereof.

Examples of alkyl groups (including those which are part of other groups), unless otherwise defined, are branched and unbranched alkyl groups with 1 to 5 carbon atoms, such as, for example: methyl, ethyl, propyl, 1-methylethyl (isopropyl), n-butyl, 1-methylpropyl, 2-methylpropyl, 1,1-dimethylethyl (tert.butyl), etc. The definitions propyl, butyl and pentyl always include the associated isomeric groups. Hydroxy or dihydroxyalkyl groups are alkyl groups substituted by one or two hydroxy groups.

Examples of alkenyl groups (including those which are part of other groups) are branched and unbranched alkenyl groups with 3 to 5 carbon atoms, provided that they have at least one double bond, such as, for example, propenyl, isopropenyl, butenyl, etc.

Cycloalkyl generally denotes a saturated cyclic hydrocarbon group having 3 to 6 carbon atoms. Examples include cyclopropyl, cyclobutyl, cyclopentyl, cyclohexyl, cyclopropylmethyl, cyclobutylmethyl, cyclopentylmethyl, cyclopropylethyl, cyclobutylethyl, etc.

Alkyloxy, which may optionally also be referred to as alkoxy, denotes a straight-chain or branched alkyl group bound via an oxygen atom. The methoxy group is particularly preferred.

Any reference to the abovementioned NK₁-receptor antagonists 2 within the scope of the present invention includes a reference to any pharmacologically acceptable acid addition salts thereof which may exist.

By the physiologically acceptable acid addition salts which may be formed from 2 are meant, for example, pharmaceutically acceptable salts selected from the salts of hydrochloric acid, hydrobromic acid, sulphuric acid, phosphoric acid, methanesulphonic acid, acetic acid, fumaric acid, succinic acid, lactic acid, citric acid, tartaric acid or maleic acid. Particularly preferred salts of the compounds 2 according to the invention are those selected from among the acetate, hydrochloride, hydrobromide, sulphate, phosphate and methanesulphonate.

The pharmaceutical combinations of 1 and 2 according to the invention are preferably administered by inhalation. Suitable inhalable powders packed into suitable capsules (inhalettes) may be administered using suitable powder inhalers. Alternatively, the drug may be inhaled by the application of suitable inhalation aerosols. These also include powdered inhalation aerosols which contain for example HFA134a, HFA227 or a mixture thereof as propellant gas. The drug may also be inhaled using suitable solutions of the pharmaceutical combination consisting of 1 and 2.

In one aspect, therefore, the invention relates to a pharmaceutical composition which contains a combination of 1 and 2.

In another aspect the present invention relates to a pharmaceutical composition which contains one or more salts 1 and one or more compounds 2, optionally in the form of their solvates or hydrates. The active substances may be combined in a single preparation or contained in two separate formulations. Pharmaceutical compositions which contain the active substances 1 and 2 in a single preparation are preferred according to the invention.

In another aspect the present invention relates to a pharmaceutical composition which contains, in addition to therapeutically effective quantities of 1 and 2, a pharmaceutically acceptable carrier or excipient. In another aspect the present invention relates to a pharmaceutical composition which does not contain any pharmaceutically acceptable excipient in addition to therapeutically effective quantities of 1 and 2.

The present invention also relates to the use of 1 and 2 for preparing a pharmaceutical composition containing therapeutically effective quantities of 1 and 2 for treating inflammatory or obstructive diseases of the respiratory tract, particularly asthma or chronic obstructive pulmonary disease (COPD), as well as complications thereof such as pulmonary hypertension, as well as allergic and non-allergic rhinitis, provided that treatment with NK₁ receptor antagonists is not contraindicated from a therapeutic point of view, by simultaneous or successive administration.

The present invention also relates to the simultaneous or successive use of therapeutically effective doses of the combination of the above pharmaceutical compositions 1 and 2 for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis, provided that treatment with NK₁ receptor antagonists is not contraindicated from a therapeutic point of view, by simultaneous or successive administration.

In the active substance combinations of 1 and 2 according to the invention, ingredients 1 and 2 may be present in the form of their enantiomers, mixtures of enantiomers or in the form of racemates.

The proportions in which the two active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. As a rule, the pharmaceutical combinations according to the invention may contain compounds 1 and 2 in ratios by weight ranging from 1:100 to 100:1, preferably from 1:80 to 80:1.

In the particularly preferred pharmaceutical combinations which contain in addition to a compound of formula 1 a compound selected from among BIIF 1149, CGP 60829, MK-869, CJ-11974, GR 205171, N-[2-(3,5-bis-trifluoromethyl-phenyl)-ethyl]-2-{4-[(3-hydroxy-propyl)-methyl-amino]-piperidin-1-yl}-N-methyl-2-phenyl-acetamide, N-[2-(3,5-bis-trifluoromethyl-phenyl)-ethyl]-2-[4-(2-hydroxy-1-hydroxymethyl-ethylamino)-piperidin-1-yl]-N-methyl-2-phenylacetamide, N-[2-(3,5-bis-trifluoromethyl-phenyl)-ethyl]-2-[4-(cyclopropylmethyl-methyl-amino)-piperidin-1-yl]-N-methyl-2-phenyl-acetamide, N-[2-(3,5-bis-trifluoromethyl-phenyl)-ethyl]-2-{4-[(2-hydroxy-ethyl)-(3-hydroxy-propyl)-amino]-piperidin-1-yl}-N-methyl-2-phenyl-acetamide and N-[2-(3,5-bis-trifluoromethyl-phenyl)-ethyl]-2-{4-[cyclopropylmethyl-(3-hydroxy-propyl)-amino]piperidin-1-yl}-N-methyl-2-phenyl-acetamide as well as the arylglycinamide derivatives of formula 3 as NK₁ receptor antagonists 2, the weight ratios of 1 to 2 are preferably in a range wherein 1′ and 2 are present in proportions ranging from 1:50 to 50:1, more preferably from 1:20 to 20:1.

For example, without restricting the scope of the invention thereto, preferred combinations of 1 and 2 according to the invention may contain the cation 1′ and NK₁ receptor antagonists 2 in the following weight ratios:

1:50; 1:49; 1:48; 1:47; 1:46; 1:45; 1:44; 1:43; 1:42; 1:41; 1:40; 1:39; 1:38; 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; 20:1.

The pharmaceutical compositions according to the invention containing the combinations of 1 and 2 are normally used so that 1 and 2 are present together in doses from 0.01 bis 10000 μg, preferably from 0.1 to 2000 μg, more preferably from 1 to 1500 μg, most preferably from 50 to 1200 μg per single dose. For example, combinations of 1 and 2 according to the invention contain an amount of 1′ and NK₁ receptor antagonist 2 such that the total dosage per single dose is 100 μg, 105 μg, 10 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 g, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg or the like. These proposed dosages per single dose are not to be regarded as being restricted to the numerical values explicitly mentioned but are merely disclosed by way of example. Obviously, dosages which fluctuate around these values within a range of about +/−2.5 μg are also covered by the values mentioned by way of example. In these dosage ranges the active substances 1′ and 2 may be present in the weight ratios described above.

For example and without restricting the scope of the invention thereto, the combinations of 1 and 2 according to the invention may contain an amount of 1′ and NK₁ receptor antagonist 2 such that 16.5 μg of 1′ and 25 μg of 2, 16.5 μg of 1 and 25 μg of 2, 16.5 μg of 1′ and 50 μg of 2, 16.5 μg of 1′ and 100 μg of 2, 16.5 μg of 1′ and 200 μg of 2, 16.5 μg of 1′ and 300 μg of 2, 16.5 μg of 1′ and 400 μg of 2, 16.5 μg of 1′ and 500 μg of 2, 16.5 μg of 1′ and 600 μg of 2, 16.5 μg of 1′ and 700 μg of 2, 16.5 μg of 1′ and 800 μg of 2, 16.5 μg of 1′ and 900 μg of 2, 16.5 μg of 1′ and 1000 μg of 33.1 μg of 1′ and 25 μg of 33.1 μg of 1′ and 50 μg of 2, 33.1 μg of 1′ and 100 μg of 2, 33.1 μg of 1′ and 200 μg of 2, 33.1 μg of 1′ and 300 μg of 2, 33.1 μg of 1′ and 400 μg of 2, 33.1 μg of 1′ and 500 μg of 2, 33.1 μg of 1 and 600 μg of 2, 33.1 μg of 1′ and 700 μg of 2, 33.1 μg of 1′ and 800 μg of 2, 33.1 μg of 1′ and 900 μg of 2, 33.1 μg of 1′ and 1000 μg of 2, 49.5 μg of 1′ and 25 μg of 2, 49.5 μg of 1′ and 50 μg of 2, 49.5 μg of 1′ and 100 μg of 2, 49.5 μg of 1 and 200 μg of 2, 49.5 μg of 1 and 300 μg of 2, 49.5 μg of 1′ and 400 μg of 2, 49.5 μg of 1′ and 500 μg of 2, 49.5 μg of 1′ and 600 μg of 2, 49.5 μg of 1′ and 700 μg of 2, 49.5 μg of 1′ and 800 μg of 2, 49.5 μg of 1′ and 900 μg of 2, 49.5 μg of 1′ and 1000 μg of 2, 82.6 μg of 1′ and 25 μg of 2, 82.6 μg of 1′ and 50 μg of 2, 82.6 μg of 1′ and 100 μg of 2, 82.6 μg of 1′ and 200 μg of 2, 82.6 μg of 1′ and 300 μg of 2, 82.6 μg of 1′ and 400 μg of 2, 82.6 μg of 1′ and 500 μg of 2, 82.6 μg of 1′ and 600 μg of 2, 82.6 μg of 1′ and 700 μg of 2, 82.6 μg of 1′ and 800 μg of 2, 82.6 μg of 1′ and 900 μg of 2, 82.6 μg of 1′ and 1000 μg of 2, 165.1 μg of 1′ and 25 μg of 2, 165.1 μg of 1′ and 50 μg of 2, 165.11 g of 1′ and 100 μg of 2, 165.1 μg of 1′ and 2001 g of 2, 165.1 μg of 1′ and 300 μg of 2, 165.1 μg of 1′ and 400 μg of 2, 165.1 μg of 1′ and 500 μg of 2, 165.1 μg of 1′ and 600 μg of 2, 165.1 μg of 1′ and 700 μg of 2, 165.1 μg of 1′ and 800 μg of 2, 165.1 μg of 1′ and 900 μg of 2, 165.1 μof 1′ and 1000 μg of 2, 206.4 μg of 1′ and 25 μg of 2, 206.4 μg of 1′ and 50 μg of 2, 206.4 μg of 1′ and 100 μg of 2, 206.4 μg of 1′ and 200 μg of 2, 206.4 μg of 1′ and 300 μg of 2, 206.4 μg of 1′ and 400 μg of 2, 206.4 μg of 1′ and 500 μg of 2 or 206.4 μg of 1′ and 600 μg of 2, 206.4 μg of 1′ and 700 μg of 2, 206.4 μg of 1′ and 800 μg of 2, 206.4 μg of 1′ and 900 μg of 2, 206.4 μg of 1′ and 100 μg of 2, 412.8 μg of 1′ and 25 μg of 2, 412.8 μg of 1′ and 50 μg of 2, 412.8 μg of 1′ and 100 μg of 2, 412.8 μg of 1′ and 200 μg of 2, 412.8 μg of 1′ and 300 μg of 2, 412.8 μg of 1′ and 400 μg of 2, 412.8 μg of 1′ and 500 μg of 2 or 412.8 μg of 1′ and 600 μg of 2, 412.8 μg of 1′ and 700 μg of 2, 412.8 μg of 1′ and 800 μg of 2, 412.8 μg of 1′ and 900 μg of 2, 412.8 μg of 1′ and 1000 μg of 2 are administered per single dose.

If the active substance combination wherein 1 denotes the bromide is used as the preferred combination of 1 and 2 according to the invention, the quantities of active substances 1′ and 2 administered per single dose as specified by way of example correspond to the following quantities of 1 and 2 administered

per single dose: 20 μg of 1 and 25 μg of 2, 20 μg of 1 and 50 μg of 2, 20 μg of 1 and 100 μg of 2, 20 μg of 1 and 200 μg of 2, 20 μg of 1 and 300 μg of 2, 20 μg of 1 and 400 μg of 2, 20 μg of 1 and 500 μg of 2, 20 μg of 1 and 600 μg of 2, 20 μg of 1 and 700 μg of 2, 201 g of 1 and 800 μg of 2, 20 μg of 1 and 900 μg of 2, 20 μg of 1 and 1000 μg of 2, 40 μg of 1 and 25 μg of 2, 40 μg of 1 and 50 μg of 2, 40 μg of 1 and 100 μg of 2, 40 μg of 1 and 200 g of 2, 40 μg of 1 and 300 μg of 2, 40 μg of 1 and 400 μg of 2, 40 μg of 1 and 500 μg of 2, 40 μg of 1 and 600 μg of 2, 40 μg of 1 and 700 μg of 2, 40 μg of 1 and 800 μg of 2, 40 μg of 1 and 900 μg of 2, 40 μg of 1 and 1000 μg of 2, 60 μg of 1 and 25 μg of 2, 60 μg of 1 and 50 μg of 2, 60 μg of 1 and 100 μg of 2, 60 μg of 1 and 200 μg of 2, 60 μg of 1 and 300 μg of 2, 60 μg of 1 and 400 μg of 2, 60 μg of 1 and 500 μg of 2, 60 μg of 1 and 600 μg of 2, 60 μg of 1 and 700 μg of 2, 60 μg of 1 and 800 μg of 2, 60 μg of 1 and 900 μg of 2, 60 μg of 1 and 1000 μg of 2, 100 μg of 1 and 25 μg of 2, 100 μg of 1 and 50 μg of 2, 100 μg of 1 and 100 μg of 2, 100 μg of 1 and 200 μg of 2, 100 μg of 1 and 300 μg of 2, 100 μg of 1 and 400 μg of 2, 100 μg of 1 and 500 μg of 2, 100 μg of 1 and 60 μg of 2, 100 μg of 1 and 700 μg of 2, 100 μg of 1 and 800 g of 2, 100 μg of 1 and 900 μg of 2, 100 μg of 1 and 1000 μg of 2, 200 μg of 1 and 25 μg of 2, 200 μg of 1 and 50 μg of 2, 200 μg of 1 and 100 μg of 2, 200 μg of 1 and 200 μg of 2, 200 μg of 1 and 300 μg of 2, 200 μg of 1 and 400 μg of 2, 200 μg of 1 and 500 μg of 2, 200 μg of 1 and 600 μg of 2, 200 μg of 1 and 700 μg of 2, 200 μg of 1 and 800 μg of 2, 200 μg of 1 and 900 μg of 2, 200 μg of 1 and 100 μg of 2, 250 μg of 1 and 25 μg of 2, 250 μg of 1 and 50 μg of 2, 250 μg of 1 and 100 μg of 2, 250 μg of 1 and 200 μg of 2, 250 μg of 1 and 300 μg of 2, 250 μg of 1 and 400 μg of 2, 250 μg of 1 and 500 μg of 2, 250 g of 1 and 600 μg of 2, 250 μg of 1 and 700 μg of 2, 250 μg of 1 and 800 μg of 2, 250 μg of 1 and 900 μg of 2, 250 μg of 1 and 1000 μg of 2, 500 μg of 1 and 25 μg of 2, 500 μg of 1 and 50 g of 2, 500 μg of 1 and 100 μg of 2, 500 μg of 1 and 200 μg of 2, 500 μg of 1 and 300 μg of 2, 500 μg of 1 and 400 μg of 2, 500 μg of 1 and 500 μg of 2, 500 μg of 1 and 600 μg of 2, 500 μg of 1 and 700 μg of 2, 500 μg of 1 and 800 μg of 2, 500 μg of 1 and 900 μg of 2 or 500 μg of 1 and 1000 μg of 2.

The active substance combinations of 1 and 2 according to the invention are preferably administered by inhalation. For this purpose, ingredients 1 and 2 have to be made available in forms suitable for inhalation. Inhalable preparations include inhalable powders, propellant-containing metering aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two or three separate formulations. These formulations which may be used within the scope of the present invention are described in more detail in the next part of the specification.

A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextran), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipients in addition to 1 and 2 are packed into capsules (to produce so-called inhalettes) which are used in inhalers as described, for example, in WO 94/28958.

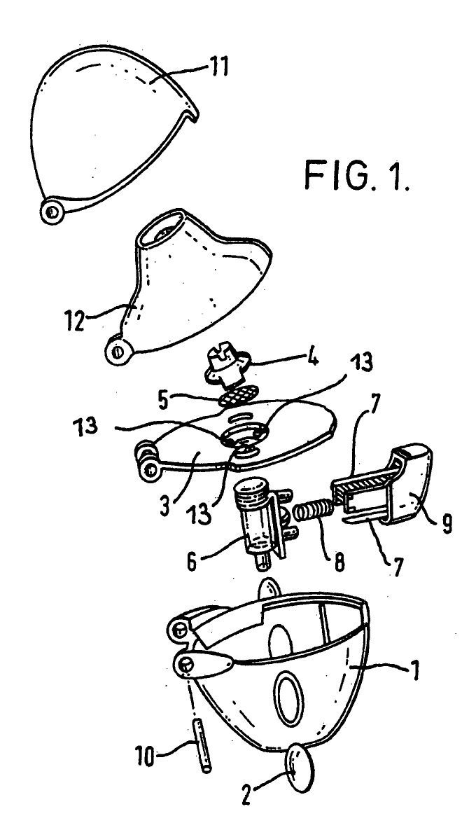

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as air through-holes 13 for adjusting the flow resistance.

If the inhalable powders according to the invention are to be packed into capsules (inhalettes) for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of 1′ and 2 mentioned hereinbefore for each single dose.

B) Propellant Gas-Driven Inhalation Aerosols Containing the Combinations of Active Substances 1 and 2 According to the Invention:

Inhalation aerosols containing propellant gas according to the invention may contain 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a and TG227. Of the abovementioned halogenated hydrocarbons, TG134a (1,1,1,2-tetrafluoroethane) and TG227 (1,1,1,2,3,3,3-heptafluoropropane) and mixtures thereof are preferred according to the invention.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the required therapeutic dose within a few seconds to produce an aerosol suitable for therapeutic inhalation. Within the scope of the present invention, preferred nebulisers are those in which a quantity of less than 100 μL, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

This nebuliser (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebuliser sprays a defined volume of the pharmaceutical formulation at high pressures through small nozzles so as to produce inhalable aerosols.

The preferred atomiser essentially consists of an upper housing part, a pump housing, a nozzle, a locking mechanism, a spring housing, a spring and a storage container, characterised by

a pump housing which is secured in the upper housing part and which comprises at one end a nozzle body with the nozzle or nozzle arrangement,

a hollow plunger with valve body,

a power takeoff flange in which the hollow plunger is secured and which is located in the upper housing part,

a locking mechanism situated in the upper housing part,

a spring housing with the spring contained therein, which is rotatably mounted on the upper housing part by means of a rotary bearing,

a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured valve bodies are disclosed for example in WO-94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 1000. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up 5 to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well, are preferably made of plastics, e.g. by injection moulding. For medicinal purposes, physiologically safe materials are used.

FIGS. 6a/b of WO 97/12687, to which reference is explicitly made at this point, show the nebuliser (Respimat®) which can advantageously be used for inhaling the aqueous aerosol preparations according to the invention. FIG. 6a of WO 97/12687 shows a longitudinal section through the atomiser with the spring biased while FIG. 6b of WO 97/12687shows a longitudinal section through the atomiser with the spring relaxed. The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the technology described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

The Examples which follow serve to illustrate the present invention in more detail without restricting the scope of the invention to the following embodiments by way of example.

### FORMULATION EXAMPLES

Inhalable powders:

| Ingredients | μg per capsule |
|---|---|
| 1) | |
| 1′-Bromide | 200 |
| N-[2-(3,5-Bis-trifluoromethyl- | 150 |
| phenyl)-ethyl]-2-{4-[(3-hydroxy- | |
| propyl)-methyl-amino]-piperidin- | |
| 1-yl}-N-methyl-2-phenyl- | |
| acetamide | |
| Lactose | 12150 |
| Total | 12500 |
| 2) | |
| 1′-Bromide | 100 |
| N-[2-(3,5-Bis-trifluoromethyl- | 125 |
| phenyl)-ethyl]-2-[4-(2-hydroxy-1- | |
| hydroxymethyl-ethylamino)- | |
| piperidin-1-yl]-N-methyl-2- | |
| phenylacetamide | |
| Lactose | 12350 |
| Total | 12500 |
| 3) | |
| 1′-Bromide | 200 |
| N-[2-(3,5-Bis-trifluoromethyl- | 250 |
| phenyl)-ethyl]-2-{4-[(3-hydroxy- | |
| propyl)-methyl-amino]-piperidin- | |
| 1-yl}-N-methyl-2-phenyl- | |
| acetamide | |
| Lactose | 12250 |
| Total | 12500 |
| 4) | |
| 1′-Bromide | 200 |
| N-[2-(3,5-Bis-trifluoromethyl- | 400 |
| phenyl)-ethyl]-2-[4- | |
| (cyclopropylmethyl-methyl- | |
| amino)-piperidin-1-yl]-N-methyl- | |
| 2-phenyl-acetamide | |
| Lactose | 24776 |
| Total | 25000 |

1) a pharmaceutical compositions, characterised in that they contain one or more anticholinergics of formula 1 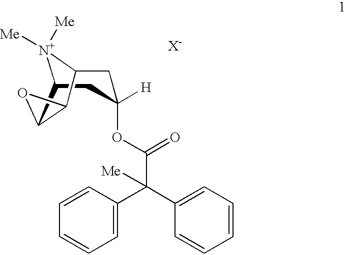
wherein
X—denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate,
combined with one or more NK₁ receptor antagonists (2), optionally in the form of the enantiomers, mixtures of the enantiomers or in the form of the racemates thereof, optionally in the form of the solvates or hydrates and optionally together with a pharmaceutically acceptable excipient.

2) The Pharmaceutical composition according to claim 1, characterised in that in the compounds of formula 1 X⁻ is a negatively charged anion selected from the group consisting of chloride, bromide, 4-toluenesulphonate and methanesulphonate.

3) The Pharmaceutical composition according to claim 1, characterised in that in the compounds of formula 1 X⁻ denotes bromide.

4) The Pharmaceutical composition according to claim 1, characterised in that 2 is selected from among BIIF 1149, CP-122721, FK-888, NKP 608C, NKP 608A, CGP 60829, SR 48968(Saredutant), SR 140333 (Nolpitantium besilate/chloride), LY 303 870 (Lanepitant), MEN-11420 (Nepadutant), SB 223412, MDL-105172A, MDL-103896, MEN-11149, MEN-11467, DNK 333A, SR-144190, YM-49244, YM-44778, ZM-274773, MEN-10930, S-19752, Neuronorm, YM-35375, DA-5018, MK-869, L-754030, CJ-11974, L-758298, DNK-33A, 6b-I, CJ-11974, TAK-637, GR 205171, N-[2-(3,5-bis-trifluoromethyl-phenyl)-ethyl]-2-{4-[(3-hydroxy-propyl)-methyl-amino]-piperidin-1-yl}-N-methyl-2-phenyl-acetamide, N-[2-(3,5-bis-trifluoromethyl-phenyl)-ethyl]-2-[4-(2-hydroxy-1-hydroxymethyl-ethylamino)-piperidin-1-yl]-N-methyl-2-phenylacetamide, N-[2-(3,5-bis-trifluoromethyl-phenyl)-ethyl]-2-[4-(cyclopropylmethyl-methyl-amino)-piperidin-1-yl]-N-methyl-2-phenyl-acetamide, N-[2-(3,5-bis-trifluoromethyl-phenyl)-ethyl]-2--{4-[(2-hydroxy-ethyl)-(3-hydroxy-propyl)-amino]-piperidin-1-yl}-N-methyl-2-phenyl-acetamide, N-[2-(3,5-bis-trifluoromethyl-phenyl)-ethyl]-2-{4-[cyclopropylmethyl-(3-hydroxy-propyl)-amino]-piperidin-1-yl}-N-methyl-2-phenyl-acetamide and the arylglycinamide derivatives of general formula 3 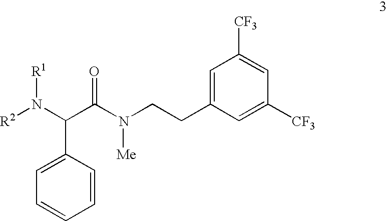
wherein
R¹ and R² together with the N to which they are bound form a ring of formula 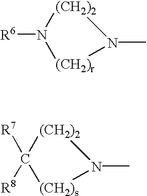
wherein r and s are 2 or 3;
R⁶ denotes H, —C₁-C₅-alkyl, C₃-C₅-alkenyl, propynyl, hydroxy(C₂-C₄)alkyl, methoxy(C₂-C₄)alkyl, di(C₁-C₃)alkylamino(C₂-C₄)alkyl, amino(C₂-C₄)alkyl, amino, di(C₁-C₃)alkylamino, monofluoro- to perfluoro(C₁-C₂)alkyl, N-methylpiperidinyl, pyridyl, pyrimidinyl, pyrazinyl or pyridazinyl,
R⁷ has one of the meanings (a) to (d),
(a) hydroxy
(b) 4-piperidinopiperidyl,
(c) 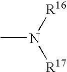
wherein R¹⁶ and R¹⁷ independently of each other denote H, (C₁-C₄)alkyl, (C₃-C₆)cycloalkyl, hydroxy(C₂-C₄)alkyl, dihydroxy(C₂-C₄)alkyl, (C₁-C₃)alkoxy(C₂-C₄)alkyl, phenyl(C₁-C₄)alkyl or di(C₁-C₃)alkylamino(C₂*C₄)alkyl,
R⁸ denotes H,
optionally in the form of the enantiomers and mixtures of enantiomers thereof, optionally in the form of the racemates thereof.

5) The Pharmaceutical composition according to claim 1, characterised in that 2 is selected from the group consisting of BIIF 1149, CP-122721, CGP 60829, MK-869, CJ-11974, GR 205171, N-[2-(3,5-bis-trifluoromethyl-phenyl)-ethyl]-2-{4-[(3-hydroxy-propyl)-methyl-amino]-piperidin-1-yl}-N-methyl-2-phenyl-acetamide, N-[2-(3,5-bis-trifluoromethyl-phenyl)-ethyl]-2-[4-(2-hydroxy-1-hydroxymethyl-ethylamino)-piperidin-1-yl]-N-methyl-2-phenylacetamide, N-[2-(3,5-bis-trifluoromethyl-phenyl)-ethyl]-2-[4-(cyclopropylmethyl-methyl-amino)-piperidin-1-yl]-N-methyl-2-phenyl-acetamide, N-[2-(3,5-bis-trifluoromethyl-phenyl)-ethyl]-2-{4-[(2-hydroxy-ethyl)-(3-hydroxy-propyl)-amino]-piperidin-1-yl}-N-methyl-2-phenyl-acetamide, N-[2-(3,5-bis-trifluoromethyl-phenyl)-ethyl]-2-{4-[cyclopropylmethyl-(3-hydroxy-propyl)-amino]-piperidin-1-yl}-N-methyl-2-phenyl-acetamide and the arylglycinamide derivatives of general formula 3, wherein
R¹ and R² together with the N to which they are bound form a ring of formula 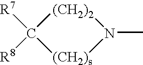
wherein s is 2 or 3;
R⁷ denotes a group 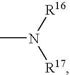
wherein R¹⁶ and R¹⁷ independently of each other denote H, (C₁-C₄)alkyl, (C₃-C₆)cycloalkyl, hydroxy(C₂-C₄)alkyl, dihydroxy(C₂-C₄)alkyl, (C₁-C₃)alkoxy(C₂-C₄)alkyl, phenyl(C₁-C₄)alkyl or di(C₁-C₃)alkylamino(C₂-C₄)alkyl,
R⁸ denotes H,
optionally in the form of the enantiomers and mixtures of enantiomers thereof and optionally in the form of the racemates thereof.

6) The Pharmaceutical compositions according to one of claim 1, characterised in that 2 is (S)-N-[2-(3,5-bis-trifluoromethyl-phenyl)-ethyl]-2-[4-(2-hydroxy-1-hydroxymethyl-ethylamino)-piperidin-1-yl]-N-methyl-2-phenylacetamide or an acid addition salt thereof.

7) The Pharmaceutical composition according to claim 1, characterised in that the weight ratios of 1 to 2 are in the range from 1:100 to 100:1, preferably from 1:80 to 80:1.

8) The Pharmaceutical composition according to claim 1, characterised in that a single administration corresponds to a dosage of the combination of active substances 1 and 2 of 0.01 to 10,000 μg, preferably from 0.1 to 2,000 μg.

9) The Pharmaceutical composition according to claim 1, characterised in that it is in the form of a formulation suitable for inhalation.

10) The Pharmaceutical composition according to claim 9, characterised in that it is a formulation selected from among inhalable powders, propellant-containing metering aerosols and propellant-free inhalable solutions or suspensions.

11) The Pharmaceutical composition according to claim 10, characterised in that it is an inhalable powder which contains 1 and 2 in admixture with suitable physiologically acceptable excipients selected from among the monosaccharides, disaccharides, oligo- and polysaccharides, polyalcohols, salts, or mixtures of these excipients.

12) The Inhalable powder according to claim 11, characterised in that the excipient has a maximum average particle size of up to 250 μm, preferably between 10 and 150 μm.

13) A Capsule, characterised in that it contains an inhalable powder according to claim 11 or 12.

14) The Pharmaceutical composition according to claim 10, characterised in that it is an inhalable powder which contains only active substances 1 and 2 as its ingredients.

15) The Pharmaceutical composition according to claim 10, characterised in that it is a propellant-containing inhalable aerosol which contains 1 and 2 in dissolved or dispersed form.

16) The Propellant-containing inhalable aerosol according to claim 15, characterised in that it contains, as propellant gas, hydrocarbons such as n-propane, n-butane or isobutane or halohydrocarbons such as chlorinated and/or fluorinated derivatives of methane, ethane, propane, butane, cyclopropane or cyclobutane.

17) The Propellant-containing inhalable aerosol according to claim 16, characterised in that the propellant gas is TG11, TG12, TG134a, TG227 or mixtures thereof.

18) The Propellant-containing inhalable aerosol according to claim 15, characterised in that it optionally contains one or more other ingredients selected from the group consisting of cosolvents, stabilisers, surfactants, antioxidants, lubricants and means for adjusting the pH.

19) The Propellant-containing inhalable aerosol according to claim 15, characterised in that it may contain up to 5 wt.-% of active substance 1 and/or 2.

20) The Pharmaceutical composition according to claim 10, characterised in that it is a propellant-free inhalable solution or suspension which contains water, ethanol or a mixture of water and ethanol as solvent.

21) The Inhalable solution or suspension according to claim 20, characterised in that the pH is 2-7, preferably 2-5.

22) The Inhalable solution or suspension according to claim 21, characterised in that the pH is adjusted by means of an acid selected from among hydrochloric acid, hydrobromic acid, nitric acid, sulphuric acid, ascorbic acid, citric acid, malic acid, tartaric acid, maleic acid, succinic acid, fumaric acid, acetic acid, formic acid and propionic acid or mixtures thereof.

23) The Inhalable solution or suspension according to claim 20, characterised in that it optionally contains other co-solvents and/or excipients.

24) The Inhalable solution or suspension according to claim 23, characterised in that it contains as co-solvents ingredients which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters.

25) The Inhalable solution or suspension according to claim 23, characterised in that it contains as excipients surfactants, stabilisers, complexing agents, antioxidants and/or preservatives, flavourings, pharmacologically acceptable salts and/or vitamins.

26) The Inhalable solution or suspension according to claim 25, characterised in that it contains as complexing agent editic acid or a salt of editic acid, preferably sodium edetate.

27) The Inhalable solution or suspension according to claim 25, characterised in that it contains, as antioxidants, compounds selected from among ascorbic acid, vitamin A, vitamin E and tocopherols.

28) The Inhalable solution or suspension according to claim 25, characterised in that it contains as preservatives compounds selected from cetyl pyridinium chloride, benzalkonium chloride, benzoic acid and benzoates.

29) The Inhalable solution or suspension according to claim 23, characterised in that it contains, in addition to the active substances 1 and 2 and the solvent, only benzalkonium chloride and sodium edetate.

30) The Inhalable solution or suspension according to claim 23, characterised in that it contains, in addition to the active substances 1 and 2 and the solvent, only benzalkonium chloride.

31) The Inhalable solution or suspension according to claim 20, characterised in that it is a concentrate or a sterile ready-to-use inhalable solution or suspension.

32) A method of nebulising in an inhaler according to WO 91/14468 or an inhaler as described in FIGS. 6a and 6b of WO 97/12687 comprising providing an inhalable solution according to claim 20.

33) The method according to to claim 31 for nebulising in an energy-operated free-standing or portable nebuliser which produces inhalable aerosols by means of ultrasound or compressed air according to the Venturi principle or other principles.

34) The Propellant-containing inhalable aerosol according to claim 17, characterised in that the propellant gas is TG134a, TG227 or a mixture thereof.

35) A Method of treatment and/or prevention of a inflammatory or obstructive diseases of the respiratory tract comprising administering to a mammal in need of such a treatment a therapeutically effective amount of a composition according to claim 1.

36) A kit comprising:
(a) a first container containing a first pharmaceutical formulation comprising one or more anticholinergics of formula 1 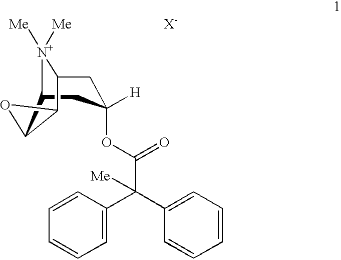
wherein
X—denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate,
optionally in the form of the enantiomers, mixtures of the enantiomers or in the form of the racemates thereof, optionally in the form of the solvates or hydrates and optionally together with a pharmaceutically acceptable excipient; and
(b) a second container containing a second pharmaceutical formulation comprising a one or more NK₁ receptor antagonists (2), optionally in the form of the enantiomers, mixtures of the enantiomers or in the form of the racemates thereof, optionally in the form of the solvates or hydrates;
each container each optionally further containing a pharmaceutically acceptable excipient.

37) A Method of treatment and/or prevention of a inflammatory or obstructive diseases of the respiratory tract comprising administering to a mammal in need of such a treatment a therapeutically effective amount of the first pharmaceutical formulation (1) comprising one or more anticholinergics of formula 1 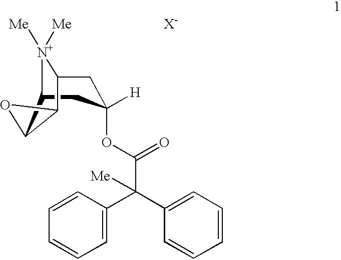
wherein
X—denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate,
and second pharmaceutical formulation comprising one or more NK₁ receptor antagonists (2),
each of (1) and (2) optionally in the form of the enantiomers, mixtures of the enantiomers or in the form of the racemates thereof, optionally in the form of the solvates or hydrates and optionally together with a pharmaceutically acceptable excipient;
wherein the first and second pharmaceutical formulations are administered simultaneously or separately.
